(19) United States
(12) Patent Application Publication
(10) Pub. No.: US 2005/0172950 A1
(43) Pub. Date: Aug. 11, 2005

(54) Low cost heated clothing manufactured from conductive loaded resin-based materials

(75) Inventor: Thomas Aisenbrey, Littleton, CO (US)

Correspondence Address: STEPHEN B. ACKERMAN, 28 DAVIS AVENUE, POUGHKEEPSIE, NY, 12603, US

(73) Assignee: Integral Technologies, Inc.

(21) Appl. No.: 11/096,176

(22) Filed: Mar. 31, 2005

Related U.S. Application Data
(63) Continuation-in-part of application No. 10/877,092, filed on Jun. 25, 2004.
Application No. 10/877,092 is a continuation of application No. 10/309,429, filed on Dec. 4, 2002, now Pat. No. 6,870,516.
Application No. 10/309,429 is a continuation-in-part of application No. 10/075,778, filed on Feb. 14, 2002, now Pat. No. 6,741,221.
(60) Provisional application No. 60/561,790, filed on Apr. 13, 2004.
(60) Provisional application No. 60/317,808, filed on Sep. 7, 2001.
(60) Provisional application No. 60/269,414, filed on Feb. 16, 2001.
(60) Provisional application No. 60/268,822, filed on Feb. 15, 2001.

(51) Int. Cl. A61F 7/00

(52) U.S. Cl. 126/208; 219/211

(57) ABSTRACT

Heated clothing devices are formed of a conductive loaded resin-based material. The conductive loaded resin-based material comprises micron conductive powder(s), conductive fiber(s), or a combination of conductive powder and conductive fibers in a base resin host. The percentage by weight of the conductive powder(s), conductive fiber(s), or a combination thereof is between about 20% and 50% of the weight of the conductive loaded resin-based material. The micron conductive powders are formed from non-metals, such as carbon, graphite, that may also be metallic plated, or the like, or from metals such as stainless steel, nickel, copper, silver, that may also be metallic plated, or the like, or from a combination of non-metal, plated, or in combination with, metal powders. The micron conductor fibers preferably are of nickel plated carbon fiber, stainless steel fiber, copper fiber, silver fiber, aluminum fiber, or the like.

This Patent Application claims priority to the U.S. Provisional Patent Application No. 60/561,790 filed on Apr. 13, 2004, which is herein incorporated by reference in its entirety.

This Patent Application is a Continuation-in-Part of INT01-002CIPC, filed as U.S. patent application Ser. No. 10/877,092, filed on Jun. 25, 2004, which is a Continuation of INT01-002CIP, filed as U.S. patent application Ser. No. 10/309,429, filed on Dec. 4, 2002, also incorporated by reference in its entirety, which is a Continuation-in-Part application of docket number INT01-002, filed as U.S. patent application Ser. No. 10/075,778, filed on Feb. 14, 2002, now issued as U.S. Pat. No. 6,741,221, which claimed priority to U.S. Provisional Patent Applications Ser. No. 60/317,808, filed on Sep. 7, 2001, Ser. No. 60/269,414, filed on Feb. 16, 2001, and Ser. No. 60/268,822, filed on Feb. 15, 2001.

### BACKGROUND OF THE INVENTION

(1) Field of the Invention

This invention relates to clothing articles and, more particularly, to clothing articles with heating elements molded of conductive loaded resin-based materials comprising micron conductive powders, micron conductive fibers, or a combination thereof, substantially homogenized within a base resin when molded. This manufacturing process yields a conductive part or material usable within the EMF or electronic spectrum(s).

(2) Description of the Prior Art

Heated clothing articles are typically manufactured in the art for use in outdoor vocational or recreational activities. Heated jackets, pants, hats, gloves, and socks, and the like, are typically made by inserting or otherwise integrating electrical heating pads into these articles. The heating pads are battery powered either using batteries carried in the clothing items or by connection to another battery source such as the electrical system of a motorcycle, a snowmobile, or the like. These heating pads are typically formed from nichrome wire that is then insulated or padded. The resulting pad is expensive to manufacture and limited in flexibility and reliability. It is a key objective of the present invention to provide a new type of clothing heater element having excellent manufacturability, flexibility, and reliability.

Several prior art inventions relate to clothing articles having conductive or heating properties. U.S. Pat. No. 4,378,226 to Tomibe et al and U.S. Pat. No. 4,364,739 to Tomibe et al teach a method to form electrically conductive fiber by impregnating copper sulfide into fiber. This fiber can then be spun and/woven into a wearable fabric. U.S. Pat. No. 5,683,744 to Jolly et al teaches a method to form a fabric with an electrically conductive polymer layer thereon. U.S. Pat. No. 5,302,807 to Zhao teaches a method of forming an electrically heated garment. The heated garment comprises synthetic fiber fabric, polyurethane foam, polyethylene film, a flexible circuit of aluminum foil, and cotton cloth all of which are stacked and glued together. The aluminum film serves as the heating element. U.S. Pat. No. 4,761,541 to Batliwalla et al teaches a laminar resistive heating element comprising an organic polymer having particulate conductive filler. The filler is disclosed as graphite or carbon black.

### SUMMARY OF THE INVENTION

A principal object of the present invention is to provide an effective clothing article.

A further object of the present invention is to provide a method to form a clothing article.

A further object of the present invention is to provide a clothing article with an integrated heating element of conductive loaded resin-based material.

A further object of the present invention is to provide a clothing article with an integrated magnetized heating element of the conductive loaded resin-based material.

A further object of the present invention is to provide a clothing article with integrated conductive circuits of the conductive loaded resin-based material.

A yet further object of the present invention is to provide a clothing article molded of conductive loaded resin-based material where the article characteristics can be altered or the visual characteristics can be altered by forming a metal layer over the conductive loaded resin-based material.

A yet further object of the present invention is to provide a method to fabricate a clothing article from a conductive loaded resin-based material where the material is in the form of a fabric.

In accordance with the objects of this invention, a heated clothing device is achieved. The device comprises a fabric covering. A heating element is integrated into the fabric covering. The conductive loaded, resin-based material comprises conductive materials in a base resin host.

Also in accordance with the objects of this invention, a heated clothing device is achieved. The device comprises a fabric covering. A heating element is integrated into the fabric covering. The heating element comprises conductive materials in a base resin host. The percent by weight of the conductive materials is between 20% and 40% of the total weight of the conductive loaded resin-based material.

Also in accordance with the objects of this invention, a heated clothing device is achieved. The device comprises a fabric covering. A heating element is integrated into the fabric covering. The heating element comprises micron conductive fiber in a base resin host. The percent by weight of the micron conductive fiber is between 20% and 40% of the total weight of the conductive loaded resin-based material.

Also in accordance with the objects of this invention, a method to form a heated clothing device is achieved. The method comprises forming a fabric covering. A conductive loaded, resin-based material is provided. The conductive loaded resin-based material comprises conductive materials in a resin-based host. The conductive loaded, resin-based material is formed into a heating element. The heating element is integrated into the fabric covering.

Also in accordance with the objects of this invention, a method to form a heated clothing device is achieved. The method comprises forming a fabric covering. A conductive loaded, resin-based material is provided. The conductive loaded resin-based material comprises conductive materials in a resin-based host. The percent by weight of the conductive materials is between 20% and 40% of the total weight of the conductive loaded resin-based material. The conductive loaded, resin-based material is formed into a heating element. The heating element is integrated into the fabric covering.

Also in accordance with the objects of this invention, a method to form a heated clothing device is achieved. The method comprises forming a fabric covering. A conductive loaded, resin-based material is provided. The conductive loaded resin-based material comprises micron conductive fiber in a resin-based host. The percent by weight of the micron conductive fiber is between 20% and 40% of the total weight of the conductive loaded resin-based material. The conductive loaded, resin-based material is formed into a heating element. The heating element is integrated into the fabric covering.

### BRIEF DESCRIPTION OF THE DRAWINGS

In the accompanying drawings forming a material part of this description, there is shown:

FIGS. 1a and 1b illustrates a first preferred embodiment of the present invention showing a garment heating element comprising a conductive loaded resin-based material.

FIG. 2 illustrates a first preferred embodiment of a conductive loaded resin-based material wherein the conductive materials comprise a powder.

FIG. 3 illustrates a second preferred embodiment of a conductive loaded resin-based material wherein the conductive materials comprise micron conductive fibers.

FIG. 4 illustrates a third preferred embodiment of a conductive loaded resin-based material wherein the conductive materials comprise both conductive powder and micron conductive fibers.

FIGS. 5a and 5b illustrate a fourth preferred embodiment wherein conductive fabric-like materials are formed from the conductive loaded resin-based material.

FIGS. 6a and 6b illustrate, in simplified schematic form, an injection molding apparatus and an extrusion molding apparatus that may be used to mold clothing article heating elements of a conductive loaded resin-based material.

FIG. 7 illustrates several additional preferred embodiments of the present invention of clothing articles comprising conductive loaded resin-based material heating elements, conductive circuits, and/or magnetic elements.

### DESCRIPTION OF THE PREFERRED EMBODIMENTS

This invention relates to heating elements for clothing articles formed of conductive loaded resin-based materials comprising micron conductive powders, micron conductive fibers, or a combination thereof, substantially homogenized within a base resin when molded.

The conductive loaded resin-based materials of the invention are base resins loaded with conductive materials, which then makes any base resin a conductor rather than an insulator. The resins provide the structural integrity to the molded part. The micron conductive fibers, micron conductive powders, or a combination thereof, are substantially homogenized within the resin during the molding process, providing the electrical continuity.

The conductive loaded resin-based materials can be molded, extruded or the like to provide almost any desired shape or size. The molded conductive loaded resin-based materials can also be cut, stamped, or vacuumed formed from an injection molded or extruded sheet or bar stock, over-molded, laminated, milled or the like to provide the desired shape and size. The thermal or electrical conductivity characteristics of clothing articles fabricated using conductive loaded resin-based materials depend on the composition of the conductive loaded resin-based materials, of which the loading or doping parameters can be adjusted, to aid in achieving the desired structural, electrical or other physical characteristics of the material. The selected materials used to fabricate the clothing articles are substantially homogenized together using molding techniques and or methods such as injection molding, over-molding, insert molding, thermo-set, protrusion, extrusion or the like. Characteristics related to 2D, 3D, 4D, and 5D designs, molding and electrical characteristics, include the physical and electrical advantages that can be achieved during the molding process of the actual parts and the polymer physics associated within the conductive networks within the molded part(s) or formed material(s).

In the conductive loaded resin-based material, electrons travel from point to point when under stress, following the path of least resistance. Most resin-based materials are insulators and represent a high resistance to electron passage. The doping of the conductive loading into the resin-based material alters the inherent resistance of the polymers. At a threshold concentration of conductive loading, the resistance through the combined mass is lowered enough to allow electron movement. Speed of electron movement depends on conductive loading concentration, that is, the separation between the conductive loading particles. Increasing conductive loading content reduces interparticle separation distance, and, at a critical distance known as the percolation point, resistance decreases dramatically and electrons move rapidly.

Resistivity is a material property that depends on the atomic bonding and on the microstructure of the material. The atomic microstructure material properties within the conductive loaded resin-based material are altered when molded into a structure. A substantially homogenized conductive microstructure of delocalized valance electrons is created. This microstructure provides sufficient charge carriers within the molded matrix structure. As a result, a low density, low resistivity, lightweight, durable, resin based polymer microstructure material is achieved. This material exhibits conductivity comparable to that of highly conductive metals such as silver, copper or aluminum, while maintaining the superior structural characteristics found in many plastics and rubbers or other structural resin based materials.

The use of conductive loaded resin-based materials in the fabrication of clothing articles significantly lowers the cost of materials and the design and manufacturing processes used to hold ease of close tolerances, by forming these materials into desired shapes and sizes. The clothing articles can be manufactured into infinite shapes and sizes using conventional forming methods such as injection molding, over-molding, or extrusion or the like. The conductive loaded resin-based materials, when molded, typically but not exclusively produce a desirable usable range of resistivity from between about 5 and 25 ohms per square, but other resistivities can be achieved by varying the doping parameters and/or resin selection(s).

The conductive loaded resin-based materials comprise micron conductive powders, micron conductive fibers, or any combination thereof, which are substantially homogenized together within the base resin, during the molding process, yielding an easy to produce low cost, electrically conductive, close tolerance manufactured part or circuit. The resulting molded article comprises a three dimensional, continuous network of conductive loading and polymer matrix. The micron conductive powders can be of carbons, graphites, amines or the like, and/or of metal powders such as nickel, copper, silver, aluminum, or plated or the like. The use of carbons or other forms of powders such as graphite(s) etc. can create additional low level electron exchange and, when used in combination with micron conductive fibers, creates a micron filler element within the micron conductive network of fiber(s) producing further electrical conductivity as well as acting as a lubricant for the molding equipment. The micron conductive fibers can be nickel plated carbon fiber, stainless steel fiber, copper fiber, silver fiber, aluminum fiber, or the like, or combinations thereof. Superconductor metals, such as titanium, nickel, niobium, and zirconium, and alloys of titanium, nickel, niobium, and zirconium may also be used as micron conductive fibers in the present invention. The structural material is a material such as any polymer resin. Structural material can be, here given as examples and not as an exhaustive list, polymer resins produced by GE PLASTICS, Pittsfield, Mass., a range of other plastics produced by GE PLASTICS, Pittsfield, Mass., a range of other plastics produced by other manufacturers, silicones produced by GE SILICONES, Waterford, N.Y., or other flexible resin-based rubber compounds produced by other manufacturers.

The resin-based structural material loaded with micron conductive powders, micron conductive fibers, or in combination thereof can be molded, using conventional molding methods such as injection molding or over-molding, or extrusion to create desired shapes and sizes. The molded conductive loaded resin-based materials can also be stamped, cut or milled as desired to form create the desired shape form factor(s) of the clothing articles. The doping composition and directionality associated with the micron conductors within the loaded base resins can affect the electrical and structural characteristics of the clothing articles and can be precisely controlled by mold designs, gating and or protrusion design(s) and or during the molding process itself. In addition, the resin base can be selected to obtain the desired thermal characteristics such as very high melting point or specific thermal conductivity.

A resin-based sandwich laminate could also be fabricated with random or continuous webbed micron stainless steel fibers or other conductive fibers, forming a cloth like material. The webbed conductive fiber can be laminated or the like to materials such as Teflon, Polyesters, or any resin-based flexible or solid material(s), which when discretely designed in fiber content(s), orientation(s) and shape(s), will produce a very highly conductive flexible cloth-like material. Such a cloth-like material could also be used in forming clothing articles that could be embedded in a person's clothing as well as other resin materials such as rubber(s) or plastic(s). When using conductive fibers as a webbed conductor as part of a laminate or cloth-like material, the fibers may have diameters of between about 3 and 12 microns, typically between about 8 and 12 microns or in the range of about 10 microns, with length(s) that can be seamless or overlapping.

The conductive loaded resin-based material of the present invention can be made resistant to corrosion and/or metal electrolysis by selecting micron conductive fiber and/or micron conductive powder and base resin that are resistant to corrosion and/or metal electrolysis. For example, if a corrosion/electrolysis resistant base resin is combined with stainless steel fiber and carbon fiber/powder, then a corrosion and/or metal electrolysis resistant conductive loaded resin-based material is achieved. Another additional and important feature of the present invention is that the conductive loaded resin-based material of the present invention may be made flame retardant. Selection of a flame-retardant (FR) base resin material allows the resulting product to exhibit flame retardant capability. This is especially important in clothing articles as described herein.

The substantially homogeneous mixing of micron conductive fiber and/or micron conductive powder and base resin described in the present invention may also be described as doping. That is, the substantially homogeneous mixing converts the typically non-conductive base resin material into a conductive material. This process is analogous to the doping process whereby a semiconductor material, such as silicon, can be converted into a conductive material through the introduction of donor/acceptor ions as is well known in the art of semiconductor devices. Therefore, the present invention uses the term doping to mean converting a typically non-conductive base resin material into a conductive material through the substantially homogeneous mixing of micron conductive fiber and/or micron conductive powder into a base resin.

As an additional and important feature of the present invention, the molded conductor loaded resin-based material exhibits excellent thermal dissipation characteristics. Therefore, clothing articles manufactured from the molded conductor loaded resin-based material can provide added thermal dissipation capabilities to the application. For example, heat can be dissipated from electrical devices physically and/or electrically connected to clothing articles of the present invention.

As a significant advantage of the present invention, clothing articles constructed of the conductive loaded resin-based material can be easily interfaced to an electrical circuit or grounded. In one embodiment, a wire can be attached to a conductive loaded resin-based clothing article via a screw that is fastened to the clothing article. For example, a simple sheet-metal type, self tapping screw, when fastened to the material, can achieve excellent electrical connectivity via the conductive matrix of the conductive loaded resin-based material. To facilitate this approach a boss may be molded into the conductive loaded resin-based material to accommodate such a screw. Alternatively, if a solderable screw material, such as copper, is used, then a wire can be soldered to the screw that is embedded into the conductive loaded resin-based material. In another embodiment, the conductive loaded resin-based material is partly or completely plated with a metal layer. The metal layer forms excellent electrical conductivity with the conductive matrix. A connection of this metal layer to another circuit or to ground is then made. For example, if the metal layer is solderable, then a soldered connection may be made between the clothing article and a grounding wire.

A typical metal deposition process for forming a metal layer onto the conductive loaded resin-based material is vacuum metallization. Vacuum metallization is the process where a metal layer, such as aluminum, is deposited on the conductive loaded resin-based material inside a vacuum chamber. In a metallic painting process, metal particles, such as silver, copper, or nickel, or the like, are dispersed in an acrylic, vinyl, epoxy, or urethane binder. Most resin-based materials accept and hold paint well, and automatic spraying systems apply coating with consistency. In addition, the excellent conductivity of the conductive loaded resin-based material of the present invention facilitates the use of extremely efficient, electrostatic painting techniques.

The conductive loaded resin-based material can be contacted in any of several ways. In one embodiment, a pin is embedded into the conductive loaded resin-based material by insert molding, ultrasonic welding, pressing, or other means. A connection with a metal wire can easily be made to this pin and results in excellent contact to the conductive loaded resin-based material. In another embodiment, a hole is formed in to the conductive loaded resin-based material either during the molding process or by a subsequent process step such as drilling, punching, or the like. A pin is then placed into the hole and is then ultrasonically welded to form a permanent mechanical and electrical contact. In yet another embodiment, a pin or a wire is soldered to the conductive loaded resin-based material. In this case, a hole is formed in the conductive loaded resin-based material either during the molding operation or by drilling, stamping, punching, or the like. A solderable layer is then formed in the hole. The solderable layer is preferably formed by metal plating. A conductor is placed into the hole and then mechanically and electrically bonded by point, wave, or reflow soldering.

Another method to provide connectivity to the conductive loaded resin-based material is through the application of a solderable ink film to the surface. One exemplary solderable ink is a combination of copper and solder particles in an epoxy resin binder. The resulting mixture is an active, screen-printable and dispensable material. During curing, the solder reflows to coat and to connect the copper particles and to thereby form a cured surface that is directly solderable without the need for additional plating or other processing steps. Any solderable material may then be mechanically and/or electrically attached, via soldering, to the conductive loaded resin-based material at the location of the applied solderable ink. Many other types of solderable inks can be used to provide this solderable surface onto the conductive loaded resin-based material of the present invention. Another exemplary embodiment of a solderable ink is a mixture of one or more metal powder systems with a reactive organic medium. This type of ink material is converted to solderable pure metal during a low temperature cure without any organic binders or alloying elements.

A ferromagnetic conductive loaded resin-based material may be formed of the present invention to create a magnetic or magnetizable form of the material. Ferromagnetic micron conductive fibers and/or ferromagnetic conductive powders are mixed with the base resin. Ferrite materials and/or rare earth magnetic materials are added as a conductive loading to the base resin. With the substantially homogeneous mixing of the ferromagnetic micron conductive fibers and/or micron conductive powders, the ferromagnetic conductive loaded resin-based material is able to produce an excellent low cost, low weight magnetize-able item. The magnets and magnetic devices of the present invention can be magnetized during or after the molding process. The magnetic strength of the magnets and magnetic devices can be varied by adjusting the amount of ferromagnetic micron conductive fibers and/or ferromagnetic micron conductive powders that are incorporated with the base resin. By increasing the amount of the ferromagnetic doping, the strength of the magnet or magnetic devices is increased. The substantially homogenous mixing of the conductive fiber network allows for a substantial amount of fiber to be added to the base resin without causing the structural integrity of the item to decline. The ferromagnetic conductive loaded resin-based magnets display the excellent physical properties of the base resin, including flexibility, moldability, strength, and resistance to environmental corrosion, along with excellent magnetic ability. In addition, the unique ferromagnetic conductive loaded resin-based material facilitates formation of items that exhibit excellent thermal and electrical conductivity as well as magnetism.

A high aspect ratio magnet is easily achieved through the use of ferromagnetic conductive micron fiber or through the combination of ferromagnetic micron powder with conductive micron fiber. The use of micron conductive fiber allows for molding articles with a high aspect ratio of conductive fiber to cross sectional area. If a ferromagnetic micron fiber is used, then this high aspect ratio translates into a high quality magnetic article. Alternatively, if a ferromagnetic micron powder is combined with micron conductive fiber, then the magnetic effect of the powder is effectively spread throughout the molded article via the network of conductive fiber such that an effective high aspect ratio molded magnetic article is achieved. The ferromagnetic conductive loaded resin-based material may be magnetized, after molding, by exposing the molded article to a strong magnetic field. Alternatively, a strong magnetic field may be used to magnetize the ferromagnetic conductive loaded resin-based material during the molding process.

Exemplary ferromagnetic conductive fiber materials include ferrite, or ceramic, materials as nickel zinc, manganese zinc, and combinations of iron, boron, and strontium, and the like. In addition, rare earth elements, such as neodymium and samarium, typified by neodymium-iron-boron, samarium-cobalt, and the like, are useful ferromagnetic conductive fiber materials. Exemplary non-ferromagnetic conductor fibers include stainless steel, nickel, copper, silver, aluminum, or other suitable metals or conductive fibers, alloys, plated materials, or combinations thereof. Superconductor metals, such as titanium, nickel, niobium, and zirconium, and alloys of titanium, nickel, niobium, and zirconium may also be used as micron conductive fibers in the present invention. Exemplary ferromagnetic micron powder leached onto the conductive fibers include ferrite, or ceramic, materials as nickel zinc, manganese zinc, and combinations of iron, boron, and strontium, and the like. In addition, rare earth elements, such as neodymium and samarium, typified by neodymium-iron-boron, samarium-cobalt, and the like, are useful ferromagnetic conductive powder materials.

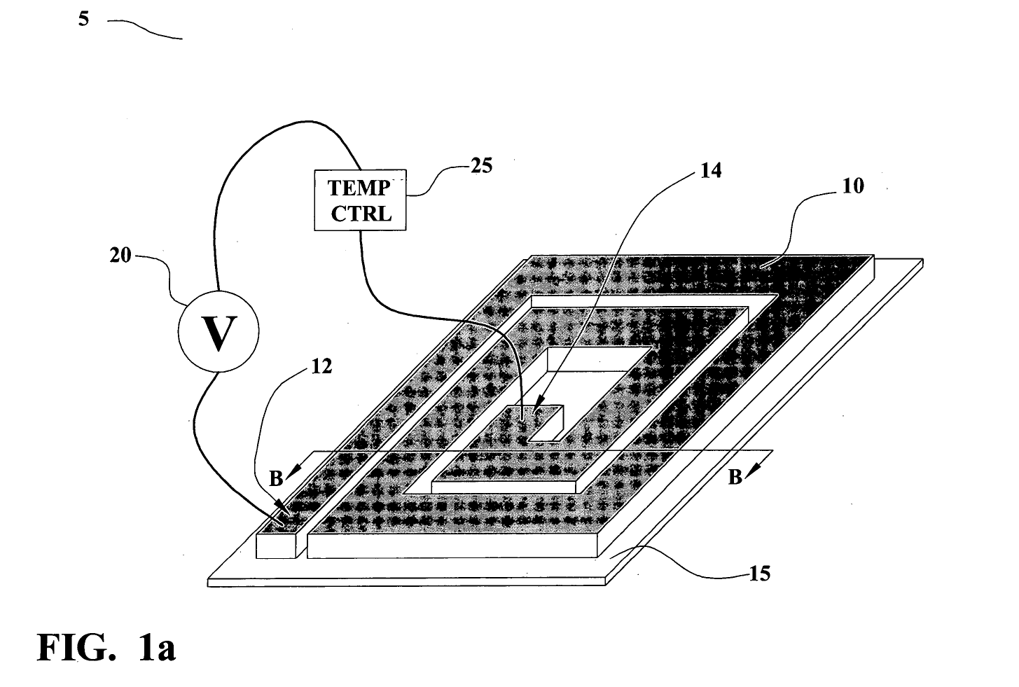

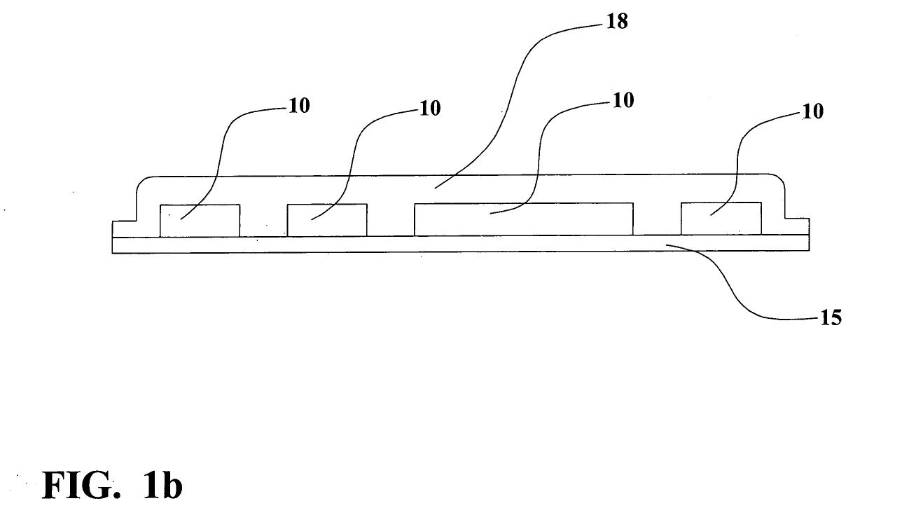

Referring now to FIGS. 1a and 1b, a first preferred embodiment 5 of the present invention is illustrated. A conductive loaded resin-based material heating element 10 for integration into a clothing article is shown. Several important features of the present invention are shown and discussed below. The resistive heating element 10 comprises a solid strip of conductive loaded resin-based material. The heating element 5 is shown connected to an electrical power source V 20. The conductive loaded resin-based material is electrically conductive. The bulk resistivity or the material can be easily adjusted by adjusting the relative amount(s) of or the type(s) of conductive materials in the base resin. The resistance of the heating element 10 is the product of the bulk resistivity of the conductive loaded resin-based material and the linear distance of the element divided by the cross sectional area of the element. The base resin material is chosen based on many factors, such as mechanical strength, flexibility, appearance, corrosion/electrolysis resistance, flame retardant characteristics, chemical characteristics, process characteristics, transparency/opaqueness, cost, etc., and on the thermal requirements of the application. For example, the glass transition temperature or maximum operating temperature of the molded resin-based material must be considered when selecting the material for a given heating element application. Very high temperature base resin materials, such as those capable of over 1000° C. operation, may be used to achieve very high temperature resistive elements of conductive loaded resin-based material according to the present invention.

The element 10 in the illustration is shaped into a spiral with an outer terminal 12 and an inner terminal 14. This arrangement is particularly useful for forming a large planar surface area for heat transfer by conduction, convection, or radiant heating. As shown in the cross section, the conductive loaded resin-based heating element 10 is preferably encased in electrically insulating material 18. In one embodiment, the spiral pattern of conductive loaded resin-based material 10 is over-molded onto an electrically insulating substrate 15. In another embodiment, the thin film of electrically insulating layer 18 is formed over the spiral pattern 10 by spraying, dipping, or the like. The top insulating layer 18 provides a non-conductive working surface that prevents electrical shock while conducting heat from the element 10 to any object in contact with the surface.

The spiral element of the present invention 10 exhibits very rapid heating and is particularly useful for heated clothing article applications. In one embodiment, the spiral element 10 is formed of a flexible material by selecting a flexible base resin in the conductive loaded resin-based material 10 and flexible resin-based insulator 18 and flexible substrate 15. In this case, the spiral element 10 will flex and is, therefore, particularly useful for direct contact applications to non-planar surfaces. The spiral element 10 may be applied to a clothing article by adhesives or by other mechanical keeps. In one embodiment, the spiral element 10 is applied to the backside of a fabric article. The spiral patterned conductive loaded resin-based heating element 10 can be modified in many ways while remaining within the scope of the present invention. While a square pattern is illustrated, any shape can by used including round, elliptical, complex perimeters, three-dimensional perimeters, parallel lines, and the like. In one embodiment, the heating element is a series of single rectangular pieces connected between terminals of a conductive bus.

Current flows from the source V 20 through the heating element 10. As the current is transmitted, heat is generated in the element 10 according to I²R. Due to the excellent thermal conductivity of the conductive loaded resin-based material, the I²R heat energy is conducted to the outer surfaces of the element. This heat energy can then be transferred away from the element 10 by conduction, convection, or radiation depending on the application and environmental conditions into which the element 10 is placed. In many clothing embodiments, a direct current (DC) from a battery source is applied to the element 10. In one embodiment, the battery source V 20 is the battery supply of a motorized vehicle such as a motorcycle or a snowmobile. In another embodiment, a portable dry cell battery is used as the power source. Alternatively, alternating current (AC) could easily be applied as would be the case if the element 10 is powered by a utility supply line such as in a residential or industrial setting. In another embodiment, a temperature control circuit 25 is added to the heating element 10 such that a controlled temperature is achieved. The temperature controller 25 preferably has a sensor element that detects the temperature of the heating element 10 and acts as a means for the controlling the current from the voltage source 20 and the voltage across the heating element 10.

The network of conductive fibers and/or powders of the conductive loaded resin-based material 10 is also present at the surface of the element. In most cases, an electrically insulating material is formed onto the conductive loaded resin-based material. In the case of radiant heat emission, the electrically insulating material 18 comprises a material selected for high transmittance of electromagnetic energy at particular wavelengths. Alternatively, if the heating element 10 is applied as a warming pad, then it would useful to form a top-side electrical insulator 18 of a material that is thermally conductive so that the heat generated by the element 10 transfers into the person wearing the clothing article. In this case, the bottom-side electrical insulator 15 should comprise a material that is both electrically and thermally insulating such that heat from the element 10 is not lost in that direction. Alternatively, the topside 18 and bottom-side electrically insulating layers 15 may comprise the same material.

The electrical insulator materials 15 and 18 include, but are not limited to, high temperature resin-based materials, metal oxides, polycarbonate materials, ceramics, and mica. The electrical insulator materials 15 and 18 may be applied by dipping, spray, coating, plating, over-molding, extrusion, adhesive application, and the like. The topside layer 18 may or may not bridge the gaps between legs of the heating element 10. If this electrically insulating layer 18 does bridge the gaps then this electrically insulating layer 18 can increase the mechanical strength and the thermal surface area of the heating element 10.

As another optional feature, a metal layer, not shown, may be formed over the surface of the conductive loaded resin-based material. The addition of a metal layer to the heating element 10 alters the electrical, thermal, visual and surface characteristics of the resulting composite structure. If the metal layer is formed directly onto the conductive loaded resin-based material 10, then this metal layer may be formed by plating or by coating. The metal layer may be formed by, for example, electroplating or physical vapor deposition. Similarly, if a resin-based material is used for the electrically insulating material, then this resin-based material is preferably one that can be metal plated as above. Additional alternative embodiments, not shown, include multiple insulating layers, embedding conductors and/or other structures in the conductive loaded resin-based material or in the electrically insulating layers and/or embedding electrically insulating layer(s) inside the conductive loaded resin-based element.

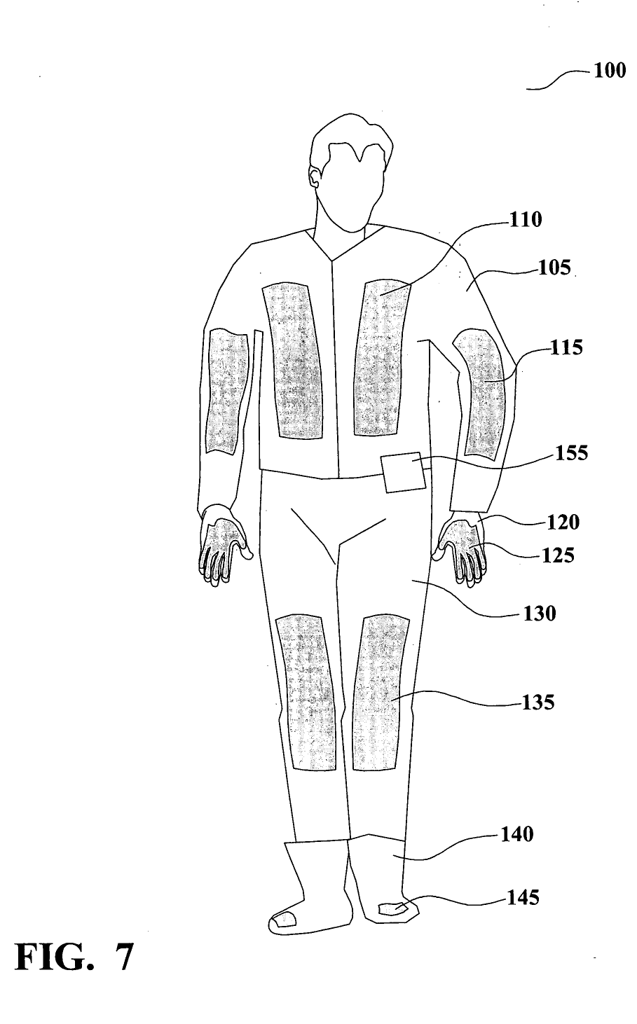

Referring now to FIG. 7, several additional preferred embodiments 100 of the present invention are illustrated. Several articles of clothing comprising the conductive loaded resin-based material of the present invention are shown. In various embodiments, a jacket 105, a pair of gloves 120, a pair of pants 130 and a pair of shoes or boots 140 are constructed to integrate conductive loaded resin-based material heating elements 110, 115, 125, 135, and 145. The heating elements are held in various locations. In one embodiment, pockets are formed in the clothing articles to hold the heating elements. In another embodiment, the heating elements are sewn into the clothing articles. In another embodiment, a temperature controller 155 is attached to the heating element. The temperature controller 155 provides a voltage source and, optionally, a temperature sensing and feedback mechanism.

In another embodiment, the heating elements 110, 115, 125, 135, and 145 are constructed as a plurality of fibers that are included in the weave of the fabric. In yet another embodiment, the heating elements 110, 115, 125, 135, and 145 are constructed as separate elements that are adhered to a woven fabric. Further, in another embodiment, the heating elements 110, 115, 125, 135, and 145 are combined with fibers formed of long molecular chains produced from poly-paraphenylene terephthalamide. One exemplary such material is commonly referred to as Kevlar™ and manufactured by DuPont, Inc., Wilmington, Del. The combination of a poly-paraphenylene terephthalamide fiber in conductive loaded resin-based material heating elements 110, 115, 125, 135, and 145 provides clothing articles offering protection as body armor combined with a heating function.

In yet another embodiment, sensors are integrated into the clothing articles 105, 120, 130, and 140. In various embodiments, sensors 110, 115, 125, 135, and 145 to monitor temperature, heart rate, and respiration rate are integrated into the articles 105, 120, 130, and 140. In one embodiment, the conductive loaded resin-based material of the present invention provides a wired connection between the sensors and a sensor pack 155. In another embodiment, the conductive loaded resin-based material provides a wireless antenna to transmit the sensor information.

In yet another embodiment, ferromagnetic conductive loading is added to the conductive loading to form ferromagnetic conductive loaded resin-based material elements 110, 115, 125, 135, and 145. In one embodiment, the magnetic elements are permanently magnetized. The elements 110, 115, 125, 135, and 145 thereby combine a heating function with a magnetic function.

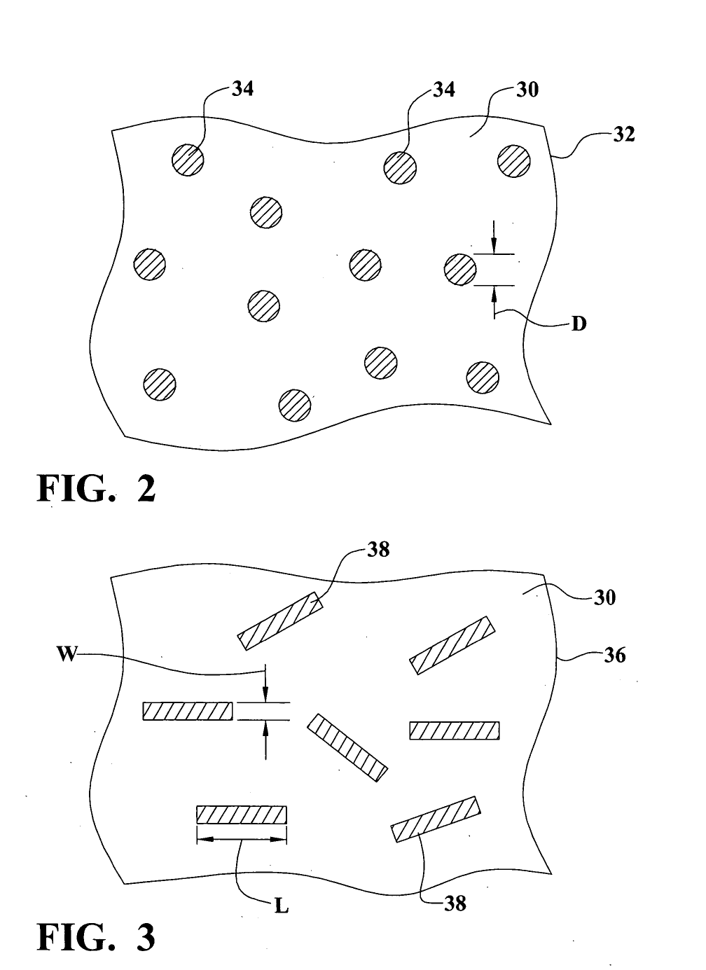

The conductive loaded resin-based material of the present invention typically comprises a micron powder(s) of conductor particles and/or in combination of micron fiber(s) substantially homogenized within a base resin host. FIG. 2 shows cross section view of an example of conductor loaded resin-based material 32 having powder of conductor particles 34 in a base resin host 30. In this example the diameter D of the conductor particles 34 in the powder is between about 3 and 12 microns.

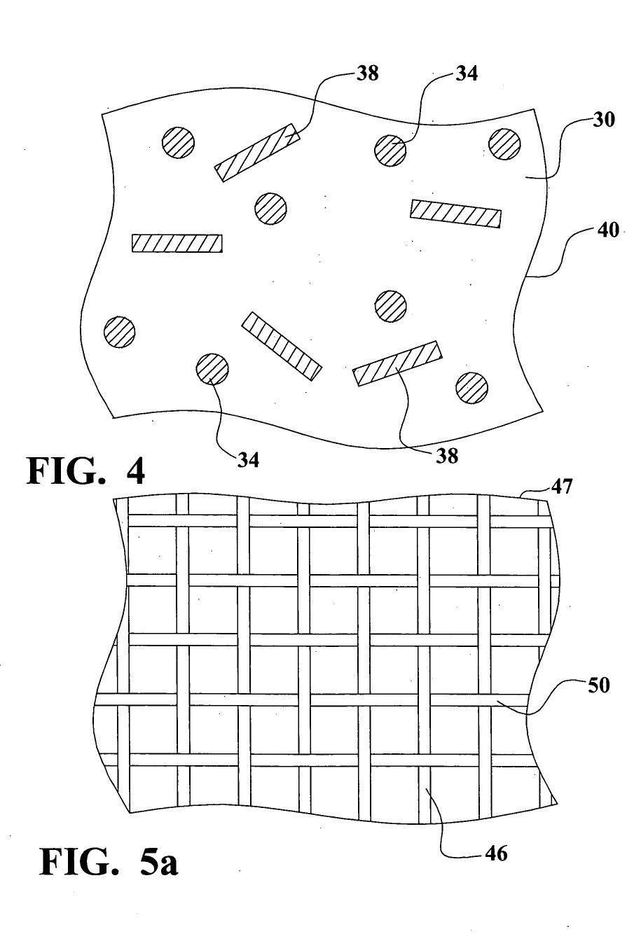

FIG. 3 shows a cross section view of an example of conductor loaded resin-based material 36 having conductor fibers 38 in a base resin host 30. The conductor fibers 38 have a diameter of between about 3 and 12 microns, typically in the range of 10 microns or between about 8 and 12 microns, and a length of between about 2 and 14 millimeters. The conductors used for these conductor particles 34 or conductor fibers 38 can be stainless steel, nickel, copper, silver, aluminum, or other suitable metals or conductive fibers, or combinations thereof. Superconductor metals, such as titanium, nickel, niobium, and zirconium, and alloys of titanium, nickel, niobium, and zirconium may also be used as micron conductive fibers in the present invention. These conductor particles and or fibers are substantially homogenized within a base resin. As previously mentioned, the conductive loaded resin-based materials have a sheet resistance between about 5 and 25 ohms per square, though other values can be achieved by varying the doping parameters and/or resin selection. To realize this sheet resistance the weight of the conductor material comprises between about 20% and about 50% of the total weight of the conductive loaded resin-based material. More preferably, the weight of the conductive material comprises between about 20% and about 40% of the total weight of the conductive loaded resin-based material. More preferably yet, the weight of the conductive material comprises between about 25% and about 35% of the total weight of the conductive loaded resin-based material. Still more preferably yet, the weight of the conductive material comprises about 30% of the total weight of the conductive loaded resin-based material. Stainless Steel Fiber of 6-12 micron in diameter and lengths of 4-6 mm and comprising, by weight, about 30% of the total weight of the conductive loaded resin-based material will produce a very highly conductive parameter, efficient within any EMF spectrum. Referring now to FIG. 4, another preferred embodiment of the present invention is illustrated where the conductive materials comprise a combination of both conductive powders 34 and micron conductive fibers 38 substantially homogenized together within the resin base 30 during a molding process.

Referring now to FIGS. 5a and 5b, a preferred composition of the conductive loaded, resin-based material is illustrated. The conductive loaded resin-based material can be formed into fibers or textiles that are then woven or webbed into a conductive fabric. The conductive loaded resin-based material is formed in strands that can be woven as shown. FIG. 5a shows a conductive fabric 42 where the fibers are woven together in a two-dimensional weave 46 and 50 of fibers or textiles. FIG. 5b shows a conductive fabric 42′ where the fibers are formed in a webbed arrangement. In the webbed arrangement, one or more continuous strands of the conductive fiber are nested in a random fashion. The resulting conductive fabrics or textiles 42, see FIG. 5a, and 42′, see FIG. 5b, can be made very thin, thick, rigid, flexible or in solid form(s).

Similarly, a conductive, but cloth-like, material can be formed using woven or webbed micron stainless steel fibers, or other micron conductive fibers. These woven or webbed conductive cloths could also be sandwich laminated to one or more layers of materials such as Polyester(s), Teflon(s), Kevlar(s) or any other desired resin-based material(s). This conductive fabric may then be cut into desired shapes and sizes.

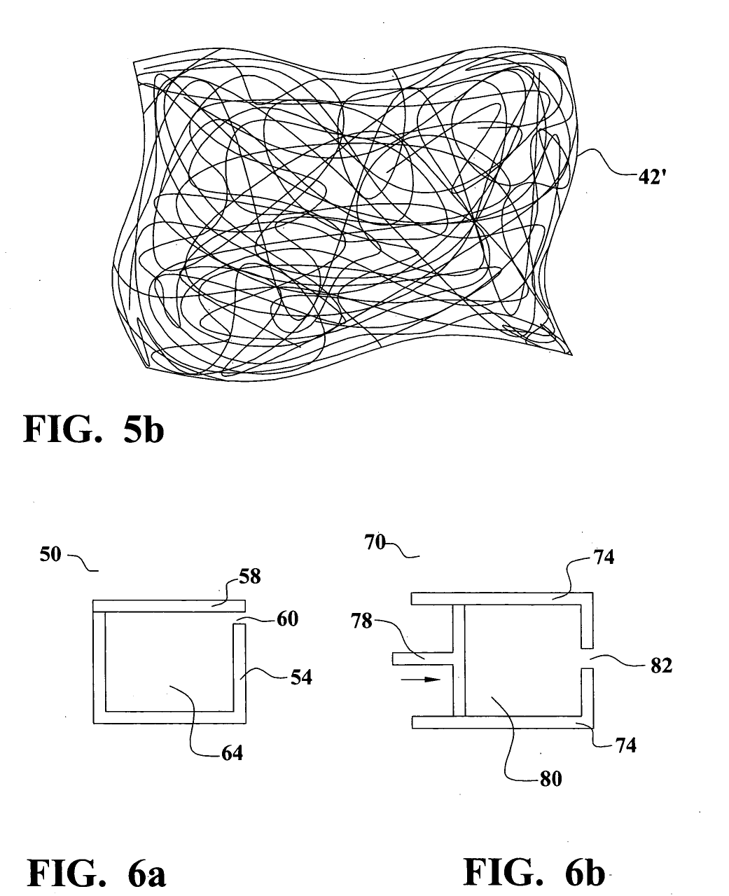

Clothing heating element devices formed from conductive loaded resin-based materials can be formed or molded in a number of different ways including injection molding, extrusion or chemically induced molding or forming. FIG. 6a shows a simplified schematic diagram of an injection mold showing a lower portion 54 and upper portion 58 of the mold 50. Conductive loaded blended resin-based material is injected into the mold cavity 64 through an injection opening 60 and then the substantially homogenized conductive material cures by thermal reaction. The upper portion 58 and lower portion 54 of the mold are then separated or parted and the heating element devices are removed.

FIG. 6b shows a simplified schematic diagram of an extruder 70 for forming heating element devices using extrusion. Conductive loaded resin-based material(s) is placed in the hopper 80 of the extrusion unit 74. A piston, screw, press or other means 78 is then used to force the thermally molten or a chemically induced curing conductive loaded resin-based material through an extrusion opening 82 which shapes the thermally molten curing or chemically induced cured conductive loaded resin-based material to the desired shape. The conductive loaded resin-based material is then fully cured by chemical reaction or thermal reaction to a hardened or pliable state and is ready for use. Thermoplastic or thermosetting resin-based materials and associated processes may be used in molding the conductive loaded resin-based articles of the present invention.

The advantages of the present invention may now be summarized. An effective clothing article is achieved. A method to form a clothing article is also achieved. The clothing article has an integrated heating element of conductive loaded resin-based material. In addition, a clothing article with an integrated magnetized heating element of the conductive loaded resin-based material is generated. In addition, a clothing article with integrated conductive circuits of the conductive loaded resin-based material. The heated clothing article is molded of conductive loaded resin-based material where the article characteristics can be altered or the visual characteristics can be altered by forming a metal layer over the conductive loaded resin-based material. A heated clothing article is formed from a conductive loaded resin-based material where the material is in the form of a fabric.

As shown in the preferred embodiments, the novel methods and devices of the present invention provide an effective and manufacturable alternative to the prior art.

While the invention has been particularly shown and described with reference to the preferred embodiments thereof, it will be understood by those skilled in the art that various changes in form and details may be made without departing from the spirit and scope of the invention.

1. A heated clothing device comprising:
a fabric covering; and
a heating element integrated into said fabric covering wherein said heating element comprises a conductive loaded, resin-based material comprising conductive materials in a base resin host.

2. The device according to claim 1 wherein the percent by weight of said conductive materials is between about 20% and about 50% of the total weight of said conductive loaded resin-based material.

3. The device according to claim 1 wherein said conductive materials comprise micron conductive fiber.

4. The device according to claim 2 wherein said conductive materials further comprise conductive powder.

5. The device according to claim 1 wherein said conductive materials are metal.

6. The device according to claim 1 wherein said conductive materials are non-conductive materials with metal plating.

7. The device according to claim 1 wherein said heating element comprises a plurality of conductive loaded resin-based material fibers woven or webbed into a conductive fabric.

8. The device according to claim 1 wherein said heating element further comprises ferromagnetic loading.

9. The device according to claim 1 wherein said fabric covering is a jacket, a shirt, a pair of pants, a sock, or a shoe.

10. A heated clothing device comprising:
a fabric covering; and
a heating element integrated into said fabric covering wherein said heating element comprises conductive materials in a base resin host and wherein the percent by weight of said conductive materials is between 20% and 40% of the total weight of said conductive loaded resin-based material.

11. The device according to claim 10 wherein said conductive materials are nickel plated carbon micron fiber, stainless steel micron fiber, copper micron fiber, silver micron fiber or combinations thereof.

12. The device according to claim 10 wherein said conductive materials comprise micron conductive fiber and conductive powder.

13. The device according to claim 12 wherein said conductive powder is nickel, copper, or silver.

14. The device according to claim 12 wherein said conductive powder is a non-conductive material with a metal plating of nickel, copper, silver, or alloys thereof.

15. The device according to claim 10 wherein said heating element comprises a plurality of conductive loaded resin-based material fibers woven or webbed into a conductive fabric.

16. The device according to claim 10 wherein said heating element further comprises ferromagnetic loading.

17. The device according to claim 10 wherein said fabric covering is a jacket, a shirt, a pair of pants, a sock, or a shoe.

18. A heated clothing device comprising:
a fabric covering; and
a heating element integrated into said fabric covering wherein said heating element comprises micron conductive fiber in a base resin host and wherein the percent by weight of said micron conductive fiber is between 20% and 40% of the total weight of said conductive loaded resin-based material.

19. The device according to claim 18 wherein said micron conductive fiber is stainless steel.

20. The device according to claim 18 wherein said micron conductive fiber has a diameter of between about 3 μm and about 12 μm and a length of between about 2 mm and about 14 mm.
